(19) United States
(12) Patent Application Publication
(10) Pub. No.: US 2023/0102930 A1
(43) Pub. Date: Mar. 30, 2023

(54) METHODS FOR PRODUCING EGGSHELL MEMBRANE HYDROLYSATES

(71) Applicants: Academia Sinica, Taipei City (TW); I-MEI FOODS CO., LTD., Taipei City (TW)

(72) Inventors: Shih-Hsiung WU, New Taipei City (TW); Ya-Chu LIEN, Yunlin County (TW); Chi-Ming KAO, New Taipei City (TW); Ken-Pei WONG, Taoyuan City (TW)

(73) Assignees: Academia Sinica, Taipei City (TW); I-MEI FOODS CO., LTD., Taipei City (TW)

(21) Appl. No.: 17/955,546

(22) Filed: Sep. 29, 2022

Related U.S. Application Data
(60) Provisional application No. 63/250,241, filed on Sep. 30, 2021.

(51) Int. Cl. C12P 21/06; C12N 1/12

(52) U.S. Cl. CPC C12P 21/06; C12N 1/12; C12R 2001/89

(57) ABSTRACT

Disclosed herein are novel methods for hydrolyzing eggshell membrane (ESM). In one embodiment, the method includes cultivating thermophilic bacteria in a solution containing 1-10% (wt %) ESM to decompose the ESM into the ESM hydrolysate; wherein, the thermophilic bacteria grow on the ESM as their sole source of nutrient. In another embodiment, the method includes treating ESM with a keratinase in the presence of a reducing agent at a condition sufficient to produce the ESM hydrolysate, in which the keratinase, the reducing agent, and the ESM are present in a weight ratio of 1:120:600. The thus produced ESM hydrolysate is enriched in essential amino acids, collagen, peptides and glycosaminoglycans.

### CROSS-REFERENCE TO RELATED APPLICATIONS

This application claims priority and the benefit of U.S. Provisional Patent Application No. 63/250,241, filed Sep. 30, 2021, the entirety of which is incorporated herein by reference.

### BACKGROUND OF THE INVENTION

### 1. Field of the Invention

The present disclosure in general relates to methods for producing eggshell membrane (ESM) hydrolysate, particularly, the method of producing the ESM hydrolysate by co-cultivating ESM with thermophilic bacteria.

### 2. Description of Related Art

The chicken eggshell membrane (ESM) is a protein-based fibrous tissue that lies between the mineralized eggshell and egg white. ESM is an exceptional biomaterial in nature with its usefulness being underestimated as it is considered as a waste material. Nevertheless, it has been found with a wide content of bioactive components and exceptional biocompatibility/biodegradability properties. In its native form, the membrane contains collagens types I, V, and X, fibronectin, proteoglycans and glycoproteins, all are essential for maintaining healthy joint and connective tissues. Further, a recent study has exploited the soluble eggshell protein fraction, extracted from raw ESM, as a possible enhancement factor to develop modified electrospun nanofibrous scaffolds.

The exploitation of ESM as source materials for biomedical uses remains overlooked and underdeveloped. Accordingly, there exist in the related art a need of an improved method for producing enriched biomaterials from ESM, which are useful in therapeutical applications (e.g., wound healing, treatment of pain and inflexibility associated with join and connective tissue disorders, and etc.) and/or as dietary supplements to prophylactically prevent pain and inflexibility associated with join and connective tissue disorders.

### SUMMARY OF THE INVENTION

The present disclosure provides novel methods for producing ESM hydrolysates that are rich in bioactive components such as essential amino acids, collagen, hyaluronic acids, and glycosaminoglycans, thus are useful in therapeutical applications and/or as dietary supplements.

In one aspect, there is provided a method of producing an ESM hydrolysate with the aid of thermophile bacteria, which secret keratinases that help digest ESM into its constituents. The method includes cultivating thermophilic bacteria in a solution containing 1-10% (wt %) ESM to decompose the ESM into desired ESM hydrolysate, wherein, the thermophilic bacteria grow on the ESM as their sole source of nutrient. Preferably, the solution contains 3% (wt %) ESM.

According to embodiments of the present disclosure, the thermophilic bacteria are thermophiles that grow at a temperature between 45° C. to 75° C. Preferably, the thermophilic bacteria are Meiothermus taiwanensis WR-220 that grow at the temperature of 55° C.

According to preferred embodiments of the present disclosure, the Meiothermus taiwanensis WR-220 are cultivated at an alkaline condition for at least 48 hrs. Preferably, the Meiothermus taiwanensis WR-220 are cultivated at pH 8.0 and 55° C. for 148 hrs.

Additionally or optionally, the method further includes adding a reducing agent to the decomposed ESM to produce the ESM hydrolysate. Examples of the reducing agent suitable for use in the present method include, but are not limited to, diborane (B₂H₆), sodium borohydride (NaBH₄), sodium sulfite (Na₂SO₃), sodium thiosulfate (Na₂S₂O₃), lithium aluminum hydride (LiAlH₄), and the like. According to preferred embodiments of the present disclosure, the reducing agent is sodium sulfite.

In another aspect, there is provided a method of producing an ESM hydrolysate by direct digesting ESM with keratinases. The method includes digesting ESM with a keratinase in the presence of a reducing agent thereby producing the ESM hydrolysate, in which the keratinase, the reducing agent, and the ESM are mixed in a weight ratio of 1:120:600.

According to embodiments of the present disclosure, the digestion is performed at pH 8.0 and 55° C. for 3 hrs.

Examples of the reducing agent suitable for use in the present method include, but are not limited to, diborane (B₂H₆), sodium borohydride (NaBH₄), sodium sulfite (Na₂SO₃), sodium thiosulfate (Na₂S₂O₃), lithium aluminum hydride (LiAlH₄), and the like.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The patent or application file contains at least one drawing executed in colors. Copies of this patent or patent application publication with color drawing(s) will be provided by the Office upon request and payment of the necessary fee.

The accompanying drawings, which are incorporated in and constitute a part of the specification, illustrate various example systems, methods and other exemplified embodiments of various aspects of the invention. The present description will be better understood from the following detailed description read in light of the accompanying drawings, where,

FIG. 1 M. taiwanensis WR-220 grow on ESM, in which (A) is the growth curve of WR-220 in the co-cultivating system; and (B) are bar graphs of free amine concentration in the ESM hydrolysate, in which ESMP stands for ESM that is grounded into powders or powdered ESM;

FIG. 2 The amino acid composition and decomposition percentage of keratinase-hydrolyzed ESM in fixed or varied concentration of keratinase or sodium sulfite, in which (A) and (B) were performed in the presence of a fixed concentration of sodium sulfite (50 mM Na₂SO₃) and varied concentration of keratinase (or “oeMtaker”); and (C) and (D) were performed in a fixed concentration of keratinase (0.650 (mM) of oeMtaker) and varied concentration of sodium sulfite; and

FIG. 3 The ratio of glycosaminoglycans and sulfated glycosaminoglycans in ESM hydrolysate, in which ESM refers to control (i.e., not treated with keratinase), ESM-EH refers to flaky ESM treated with keratinase, and ESMP-EH refers to powdered ESM treated with keratinase.

### DETAILED DESCRIPTION OF THE PREFERRED EMBODIMENTS

The detailed description provided below in connection with the appended drawings is intended as a description of the present disclosure and is not intended to represent the only forms in which the present disclosure may be constructed or utilized.

### 1. Decomposing ESM via Co-Cultivating ESM with Thermophile Bacteria

The first aspect of the present disclosure is directed to a method of producing ESM hydrolysate with the aid of thermophile bacteria, which secret keratinases that help digest ESM into its constituents. The method thus includes cultivating thermophilic bacteria in a solution containing ESM at a condition suitable for decomposing ESM into the desired ESM hydrolysate, in which the thermophilic bacteria grow on the ESM as their sole source of nutrient.

According to embodiments of the present disclosure, the thermophilic bacteria are cultivated in an ESM solution under an alkaline condition for at least 48 hrs. Preferably, the solution contains 1-10% ESM by weight, such as 1, 2, 3, 4, 5, 6, 7, 8, 9 or 10% ESM by weight; more preferably, the solution contains 2-8% ESM by weight, such as 2, 3, 4, 5, 6, 7, or 8% ESM by weight; most preferably, the solution contains 3% ESM by weight. According to embodiments of the present disclosure, the thermophilic bacteria are cultivated in 3% (wt %) ESM solution at pH 7.5 to 8.5 for at least 48 hrs, such as at pH 7.5, 7.6, 7.7, 7.8, 7.9, 8.0, 8.1, 8.2, 8.3, 8.4 or 8.5 for a period of 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81/ 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 hrs. Preferably, the thermophilic bacteria are cultivated in 3% (wt %) ESM solution at pH 8.0 for at least 148 hrs.

According to embodiments of the present disclosure, the thermophilic bacteria are thermophiles that grow at a temperature between 45° C. to 75° C. Examples of thermophilic bacteria suitable for use in the present method include, but are not limited to, Thermus aquaticus, Thermus igniterrae, Bacillus stearothermophilus, Meiothermus taiwanensis WR-220, Meiothermus ruber, Meiothermus cerbereus, and etc. According to preferred embodiments of the present disclosure, the thermophilic bacteria are Meiothermus taiwanensis WR-220, which grow on ESM at the temperature of 55° C.

According to preferred embodiments of the present disclosure, the Meiothermus taiwanensis WR-220 are cultivated in a solution containing 3% (wt %) ESM at pH 8.0 for at least 148 hrs.

Additionally, or optionally, the method further includes adding a reducing agent to the decomposed ESM to produce the ESM hydrolysate. Examples of the reducing agent suitable for use in the present method include, but are not limited to, diborane (B₂H₆), sodium borohydride (NaBH₄), sodium sulfite (Na₂SO₃), sodium thiosulfate (Na₂S₂O₃), lithium aluminum hydride (LiAlH₄), and the like. According to preferred embodiments of the present disclosure, the reducing agent is sodium sulfite.

The ESM hydrolysate produced by the present method is enriched with essential amino acids (e.g., glutamine, glutamic acid, cysteine, cystine, serine, methionine, glycine and etc), glycosaminoglycans (e.g., hyaluronic acid, chondroitin), and collagen peptides, of which type X collagen is the main one. Accordingly, the ESM hydrolysate may be used for the development of medicaments, dietary healthy supplements, and/or cosmetic products.

### 2. Decomposing ESM via Hydrolyzing ESM with Keratinases

Alternatively, instead of co-cultivating ESM with thermophilic bacteria as described above, the ESM hydrolysate may be produced by direct digesting ESM with keratinases. Accordingly, the second aspect of the present disclosure is directed to a method of producing ESM hydrolysate by digesting ESM with a keratinase in the presence of a reducing agent, in which the keratinase, the reducing agent and the ESM are respectively present in a ratio of 1:120:600 by weight.

According to embodiments of the present disclosure, the digestion is performed at pH 7.5 to 8.5 under a temperature of at least 50° C. in the presence of a reducing agent for at least 3 hrs, such as at pH 7.5, 7.6, 7.7, 7.8, 7.9, 8.0, 8.1, 8.2, 8.3, 8.4 or 8.5, at the temperature of 50, 51, 52, 53, 54, or 55° C. in the presence of a reducing agent for a period of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81/ 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 hrs. Examples of the reducing agent suitable for use in the present method include, but are not limited to, diborane (B₂H₆), sodium borohydride (NaBH₄), sodium sulfite (Na₂SO₃), sodium thiosulfate (Na₂S₂O₃), lithium aluminum hydride (LiAlH₄), and the like. According to preferred embodiments of the present disclosure, the reducing agent is sodium sulfite.

Preferably, the ESM solution (e.g., 3% (wt %) ESM) is treated with a keratinase, which may be any commercially available keratinase, at pH 8.0 and 55° C. in the presence of sodium sulfite for 3 hrs.

The ESM hydrolysate produced by the present method is enriched with essential amino acids (e.g., glutamine, glutamic acid, cysteine, cystine, serine, methionine, glycine and etc), glycosaminoglycans (e.g., hyaluronic acid, chondroitin), and collagen peptides, of which type X collagen is the main one. Accordingly, the ESM hydrolysate may be used for the development of medicaments, dietary healthy supplements, and/or cosmetic products.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

### Materials and Methods

### Eggshell Membrane (ESM) Extraction

Fresh eggs were washed carefully with distilled water before incubating them at room temperature, submerged in 0.5 M acetic acid for 44 hrs. After complete dissolution of the calcium carbonate shell, the extracted membranes were collected and washed in distilled water thoroughly to remove the albumen and York. All extracted ESM were fully immersed in PBS to avoid dehydration and stored in a refrigerator (4° C.) before use.

### Bacterial Culture

The strain M. taiwanensis WR-220 (ATCC BAA-400) were grown under aerobic conditions at 55° C. in Thermus modified (TM) medium at pH 8.0, 55° C. under constant agitation at a speed of 190 rpm, and usually sub-cultured twice before start-up ESM fermentation.

### Production of Microorganism-Based ESM hydrolysate (ESM-M.t.)

Microorganism-based ESM hydrolysate (ESM-M.t.) was produced by submerged ESM fermentation, which was initiated by adding ESM (3 g) or ESMP (3 g) as the sole carbon and nitrogen source to the cultivated M. taiwanensis WR-220 basal media (100 mL) in sterilized conical flasks (250 mL). The cultures were incubated at pH 8.0, 55° C. with constant shaking at a speed of 190 rpm. After 24, 48, and 144 hr incubation, the samples (2 mL) were harvested for cell density determination (0D600), and free amine concentration determination.

### Optimization of the ESM Enzymatic Hydrolysis (ESM-EH) Condition by Keratinases

The purified keratinase heterologous overexpressed in E. coli was named oeMtaker. To determine the concentration dependence of oeMtaker on ESM solubilization, a test solution of 30 mg ESMP, 50 μL of 1 M Na₂SO₃ in 1 mL keratinase buffer (30 mM HEPES, 250 mM NaCl, pH 8.0) was used. Incubation was carried out under conditions in which the final concentrations were 50 mM Na₂SO₃ and 0, 0.1625, 0.3650, 0.650 and 1.300 (μM) oeMtaker (12.3±0.7 U). In other words, the weight ratio of the corresponding ESM is 0:30, 1:6000, 1:3000, 1:600, and 1:300. For sodium sulfite concentration dependence of ESM solubilization, the test solution was 30 mg of ESMP, 0.05 mg of oeMtaker in 1 mL keratinase buffer. Incubation was carried out under conditions that the final concentrations were 0.650 μM oeMtaker and 0, 10, 20, 30, 50, 100 and 200 mM Na₂SO₃. That is, the weight ratio of the corresponding ESM was 0:30, 1:23.1, 1:12.0, 1:7.9, 1:4.8, 1:2.4, and 1:1.2. Each sample was subjected to hydrolysis reaction at 55° C., with 1000 rpm shaking for 3 h. After incubation, each ESM-EH sample was harvested by centrifugation at 17,000×g, 4° C. for 10 min. The supernatant and remaining ESM were collected for further experiments.

### Decomposition Percentage Measurement of ESM in Keratinase-Mediated ESM Hydrolysis System

After each reaction, 2 mL of double distilled (DD) water (repeated twice) was added to wash the remaining ESM. Then, the residual ESM was placed in a 55° C. oven to dry. After cooling to room temperature, it was weighed. The decomposition percentage (%) of ESM by the keratinase-mediated ESM decomposition system was calculated using the following equation (1).

$$\frac{Original ESM weight ( mg ) - Residual ESM weight ( mg )}{Original ESM weight ( mg )} \times 100 \% \qquad ( 1 )$$

### Concentration Measurement of Free Amines in the ESM-EH

The concentration of free amines of the ESM-EH was quantified by the ninhydrin colorimetric method. ESM-EH supernatant (100 μL) was mixed with 50 μL of ninhydrin reagent. The reaction mixture was incubated at 100° C. for 10 min, and then 75 μL of the sample was loaded into a 96-microwell plate as it was cooling. The reaction mixture was mixed well with 125 μL of 95% ethanol for subsequent absorbance measurement at 570 nm. Glycine was used as a standard.

### Analysis of Amino Acid Composition in the ESM-EH

A TRAQ Kit was used to perform amino acid composition and quantitative analysis by Liquid Chromatography Mass Spectrometry (LC-MS). The sample preparation used 0.650 μM Mtaker with 50 mM Na₂SO₃ to hydrolyze the ESM at 55° C. for 3 hr. After hydrolization, the upperpart of the ESM-EH was filtered by a 0.22 μm PVDF filter membrane and then calibrated according to the original experimental procedure of the TRAQ Kit (Boemer et al., Ann. Biol. Clin. (2015) 73, 427-442).

### Determination of Glycosaminoglycans (GAGs) and Sulfated GAGs Concentration

ESM-EH (1 mL) was purified using Bio-Gel P-6 column chromatography. Fractions that contained the sugar were collected from the ESM-EH and samples were obtained after lyophilization. The concentration of GAGs in the hydrolysate was by the carbazole method. Briefly, the organic components were released after hydrolysis of GAGs by sulfuric acid and then detected with carbazole reagent which can be observed at 530 nm. The reaction mixture contained 80 μL of sample and 400 μL of 0.025 M sodium tetraborate in sulfuric acid. The mixture was incubated at 100° C. for 15 min and then mixed with 0.125% (w/v) of carbazole in absolute ethanol (16 μL) after cooling to room temperature. The mixed solution was incubated for another 15 min at 100° C. After cooling to room temperature, its absorbance was detected at 530 nm. GAGs were quantified based on the calibration line of sodium hyaluronate. The concentration of sulfated GAGs in ESM-EH was determined by the dimethylmethylene blue assay (DMMB). This method uses dimethylmethylene blue to react with sulfated GAGs (such as chondroitin sulfate, dermatan sulfate, etc.) to change the absorption value of the reagent. After adding 10 μL of sample to 100 μL of DMMB reagent, the absorbance was measured at a specific wavelength of 525 nm within 10 min.

### Example 1 Preparation of ESM Hydrolysate (ESM-M.t.) by Co-Cultivating M. taiwanensis WR-220 in ESM

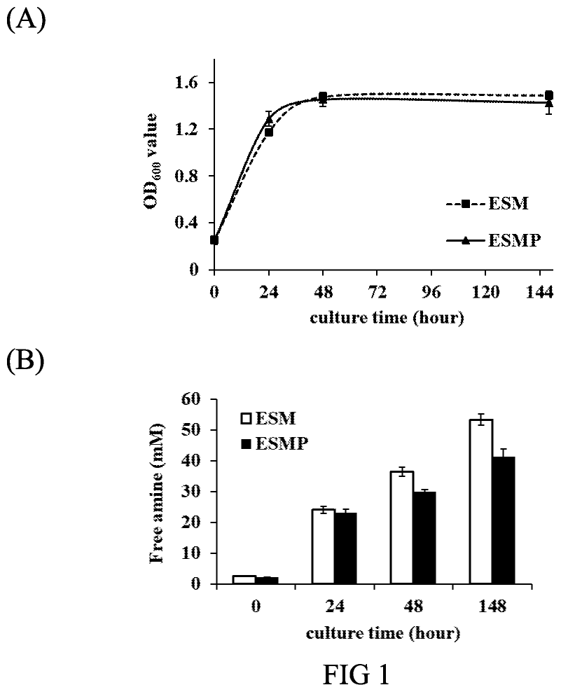

In this example, ESM hydrolysates (ESM-M.t.) were produced by cultivating M. taiwanensis WR-220 in 3% ESM or ESMP at pH 8.0, 55° C. under constant agitation at a speed of 190 rpm for 148 hrs. Bacteria growth reached a plateau after 48 hrs in culture, and remained at the plateau phase for additional 100 hrs (i.e., 148 hrs in total) (FIG. 1, (A)), an indication that M. taiwanensis WR-220 could grow on ESM or powdered ESM (ESMP) as their sole source of nutrients. At the same time, the concentration of free amine in the bacteria growth medium containing ESM or ESMP continued to increase (FIG. 1, (B)), an indication that ESM was being digested by the keratinase secreted from M. taiwanensis WR-220. As the concentration of free amine measured from the ESMP containing growth medium was lower than that from the ESM containing growth medium, we postulated that ESMP could be consumed by the bacteria in a more efficient manner as they were smaller in size than that of ESM.

### Example 2 Preparation of ESM Hydrolysate (ESM-EH) by Digesting ESM with Keratinases

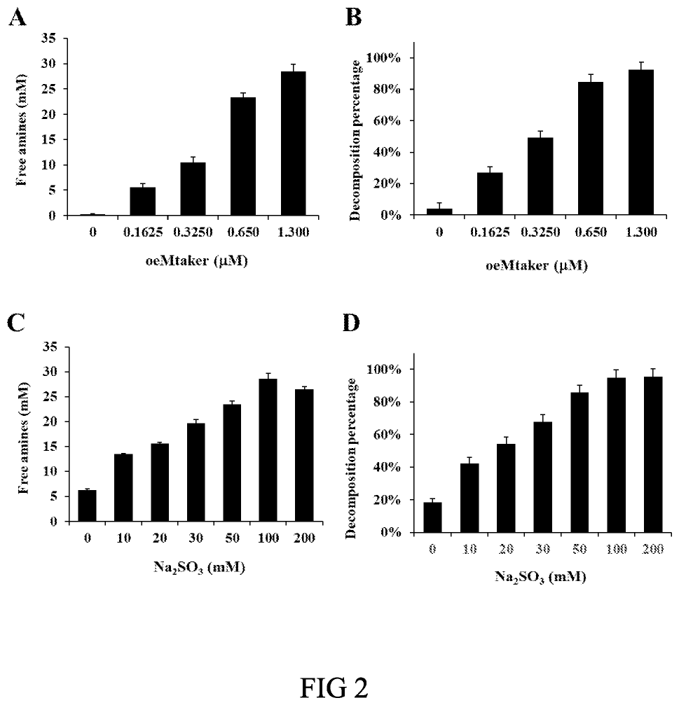

In this example, ESM hydrolysates (ESM-EH) were produced by direct digesting ESM with keratinases (i.e., oeMtaker). To this purpose, ESM was treated with fixed or varied concentrations of keratinases (0.1625 to 1.300 μM) in the presence of fixed or varied concentrations of Na₂SO₃ (0 to 200 mM) at 55° C. for 3 hrs, and results are depicted in FIG. 2.

It was found that free amine concentrations in the hydrolysate and the decomposition percentage of ESM respectively increased with the increase in the concentration of keratinase (FIG. 2, (A) and (B)) or Na₂SO₃ (FIG. 2, (C) and (D)). The results also confirmed that while hydrolyzing ESM, the addition of a reducing agent (i.e., sodium sulfite) could enhance the enzymatic activity of keratinase.

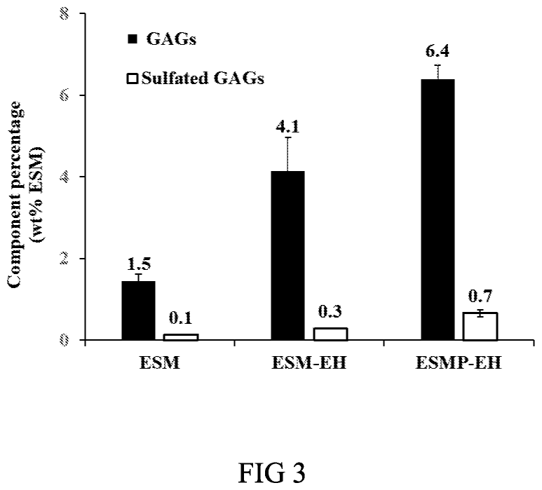

The thus produced ESM-EH was further subjected to LC-MS analysis, and the results indicated the ESM-EH was enriched in essential amino acids (data not shown), glycosaminoglycans (GAGs), sulfated GAGs, and collagen peptides (data not shown), of which type X collagen was the main one. Further, it was found that one gram of ESM could produce 4.1-6.4% GAGs and 0.3-0.7% sulfated GAGs (Table 1, and FIG. 3).

TABLE 1
Decomposition percentage and components of ESM
hydrolysate digested with keratinases (ESM-EH)

| | Decom- | Free | | |
|---|---|---|---|---|
| | position % | amines | GAGs | Sulfated GAGs |
| | (wt % ESM) | (mM) | (wt % ESM) | (wt % ESM) |
| ESM-EH | 81.5 ± 2.5 | 24.6 ± 1.1 | 6.4 ± 0.3 | 0.7 ± 0.1 |

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

What is claimed is:

1. A method of producing an eggshell membrane (ESM) hydrolysate comprising:
cultivating thermophilic bacteria in a solution containing 1-10% (wt %) ESM; wherein,
the thermophilic bacteria grow on the ESM in the solution as their sole source of nutrients thereby decomposing the ESM into the ESM hydrolysate.

2. The method of claim 1, wherein the solution contains 3% (wt %) ESM.

3. The method of claim 1, wherein the thermophilic bacteria are any one of Thermus aquaticus, Thermus igniterrae, Bacillus stearothermophilus, Meiothermus taiwanensis WR-220, Meiothermus ruber or Meiothermus cerbereus that grow at a temperature between 45° C. to 75° C.

4. The method of claim 3, wherein the thermophilic bacteria are Meiothermus taiwanensis WR-220 that grow at the temperature of 55° C.

5. The method of claim 4, wherein the Meiothermus taiwanensis WR-220 are cultivated at an alkaline condition for at least 48 hrs.

6. The method of claim 5, wherein the Meiothermus taiwanensis WR-220 are cultivated at pH 8.0 for 148 hrs.

7. The method of claim 1, further comprising adding a reducing agent to the decomposed ESM to produce the ESM hydrolysate.

8. The method of claim 7, wherein the reducing agent is selected from the group consisting of diborane (B₂H₆), sodium borohydride (NaBH₄), sodium sulfite (Na₂SO₃), sodium thiosulfate (Na₂S₂O₃), and lithium aluminum hydride (LiAlH₄).

9. The method of claim 8, wherein the reducing agent is sodium sulfite.

10. A method of producing an eggshell membrane (ESM) hydrolysate comprising,
digesting ESM with a keratinase in the presence of a reducing agent at a condition sufficient to produce the ESM hydrolysate,
wherein, the keratinase, the reducing agent, and the ESM are mixed in a weight ratio of 1:120:600.

11. The method of claim 10, wherein the condition has a pH value of 8.0, a temperature of 55° C., and a time period of 3 hrs.

12. The method of claim 10, wherein the reducing agent is selected from the group consisting of diborane (B₂H₆), sodium borohydride (NaBH₄), sodium sulfite (Na₂SO₃), sodium thiosulfate (Na₂S₂O₃), and lithium aluminum hydride (LiAlH₄)
